(19) United States
(12) Patent Application Publication
(10) Pub. No.: US 2007/0025947 A1
(43) Pub. Date: Feb. 1, 2007

(54) Anti-acne method and kit

(75) Inventors: Isabelle Hansenne, Westfield, NJ (US); Hani Fares, Somerset, NJ (US); Marc Cornell, Jackson, NJ (US); Sidney Peter Foltis, Nutley, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: L'OREAL, Paris (FR)

(21) Appl. No.: 11/192,721

(22) Filed: Jul. 29, 2005

(51) Int. Cl. A61K 8/368; A61K 8/365

(52) U.S. Cl. 424/70.22

(57) ABSTRACT

A cosmetic kit for treating human skin comprising a multi-unit receptacle containing: (a) at least one unit containing a skin preparatory composition containing at least one skin peeling agent; (b) at least one unit containing a keratolytic composition containing at least one anti-acne agent; (c) at least one unit containing a skin soothing composition; (d) optionally, at least one unit containing a maintenance composition having at least one agent chosen from a skin peeling agent and an anti-acne agent; (e) optionally, at least one unit containing a photoprotective composition.

### BACKGROUND OF THE INVENTION

Skin disorders, such as acne, can be irritating to the skin and embarrassing to the person suffering from the disorder. This is the most common reason for a visit to a dermatologist. There are many treatments, but no cure for acne. These include antibiotics (which inhibit the growth of Propionobacter acnes bacteria which play a role in acne), retinoids such as Accutane® or Differin® (which reduce sebaceous gland output of sebum), and anti-microbials such as benzoyl peroxide, α-hydroxy acids or β-hydroxy acids. Acne lesions result from the rupture of a sebaceous follicle, followed by inflammation and pus (a “whitehead”), or by accumulation of plugged material in the sebaceous follicle (a “blackhead”). It is thus important to keep the skin clean, and provide the lesions with active agents. Unfortunately, cleansing is not always sufficient. Furthermore, the active agents used for the treatment of acne tend to be harsh and irritating. Furthermore, the more powerful actives may need to be applied by a dermatologist.

Thus, there is a need for a kit comprising a multi-unit receptacle which houses all the components needed for treatment of acne and a need for a method of treatment which is simple, effective and less irritating and does not require visits to a dermatologist for treatment.

### BRIEF SUMMARY OF THE INVENTION

The present invention relates to a cosmetic kit for treating human skin comprising a multi-unit receptacle containing:

(a) at least one unit containing a skin preparatory composition containing at least one skin peeling agent;

(b) at least one unit containing a keratolytic composition containing at least one anti-acne agent;

(c) at least one unit containing a skin soothing composition having at least one skin soothing agent;

(d) optionally, at least one unit containing a maintenance composition having at least one agent chosen from a skin peeling agent and an anti-acne agent; and

(e) optionally, at least one unit containing a photoprotective composition.

According to another embodiment of the present invention, there is provided a method for treating skin, intended to treat the signs of acne, involving the steps of:

(a) providing a unit containing a skin preparatory composition containing at least one skin peeling agent;

(b) providing a unit containing a keratolytic composition containing at least one anti-acne agent;

(c) providing a unit containing a skin soothing composition having at least one skin soothing agent;

(d) optionally, providing a unit containing a photoprotective composition, followed by, in succession;

(e) contacting the skin with the skin preparatory composition;

(f) applying a layer of keratolytic composition over the skin preparatory composition;

(g) applying a layer of skin soothing composition over the skin preparatory composition and the keratolytic composition;

(h) allowing the skin preparatory composition, keratolytic composition and skin soothing composition to remain on the skin for a predetermined period of time;

(i) removing the skin preparatory composition, keratolytic composition, and skin soothing composition from the skin; and

(j) applying the photoprotective composition onto the skin.

This method is used for an amount of time sufficient for treating the acne-effected area.

### DETAILED DESCRIPTION OF THE INVENTION

Other than in the operating examples, or where otherwise indicated, all numbers expressing quantities of ingredients and/or reaction conditions are to be understood as being modified in all instances by the term “about”.

The Skin Preparatory Composition

The skin preparatory composition contains at least one type of skin peeling agent. Suitable skin peeling agents include, but are not limited to: (i) hydroxy acids such as α-hydroxy acids, such as citric, lactic, glycolic, malic, tartaric which are hydrophilic, or mandelic acid, which is lipophilic; β-hydroxy acids, such as salicylic acid which is hydrophilic; and lipophilic derivatives of hydroxy acids such as 5-(n-octanoyl)salicylic acid (also known as Capryloyl Salicylic Acid under the CTFA designation), examples of which are described in U.S. Pat. No. 4,767,750, the entire contents of which is hereby incorporated by reference; (ii) urea; (iii) aminosulphonic compounds such as N-(2-hydroxyethyl)piperazine-N′-2-ethanesulphonic acid (HEPES); (iv) derivatives of 2-oxothiazolidine-4-carboxylic acid (procysteine). In a particularly preferred embodiment, the skin preparatory composition contains a combination of salicylic acid and capryloyl salicylic acid.

The skin peeling agents may be present in the skin preparatory composition in an amount sufficient to exfoliate the top layer of the skin to remove dead skin cells, correct surface skin imperfections and increase epidermal permeability. The skin peeling agent may preferably be present in the skin preparatory composition in an amount of from 3 to 20% by weight, preferably from 5 to 15% by weight, and more preferably from 5 to 10% by weight, based on the total weight of the skin preparatory composition.

The skin preparatory composition may also contain at least one anti-acne agent conventionally used for treating acne such as, for example, salicylic acid.

According to one embodiment of the invention, the skin preparatory composition contains 0.5% by weight of capryloyl salicylic acid and 1.5% by weight of salicylic acid, all weights based on the total weight of the skin preparatory composition. In a particularly preferred embodiment, the skin preparatory composition is contained in a plurality of single-dose units, more preferably 11 single-dose units. The precise number of single-dose units contained in the cosmetic kit of the present invention will depend on the length of the treatment.

The skin preparatory composition may be in the form of a lotion, gel, fluid or cream. In an alternative form, it can be impregnated onto a wipe or cotton pad.

The Keratolytic Composition

The keratolytic composition contains at least one anti-acne agent. Suitable anti-acne agents include, but are not limited to, resorcinol, sulfur, salicylic acid, retinoids such as retinoic acid and its derivatives, sulfur-containing amino acids and their derivatives and salts, particularly their N-acetyl derivatives, an example of which may be N-acetyl-L-cysteine; and lipoic acid. Other suitable anti-acne agents may be chosen from (i) antibiotics and antimicrobials such as benzoyl peroxide, octopirox, tetracycline, 2,4,4′-trichloro-2′-hydroxy diphenyl ether, 3,4,4′-trichlorobanilide, azelaic acid and its derivatives, phenoxyethanol, phenoxypropanol, phenoxyisopropanol, ethyl acetate, clindamycin and meclocycline; (ii) sebostats such as flavonoids; and (iii) bile salts such as scymnol sulfate and its derivatives, deoxycholate, and cholate. In a particularly preferred embodiment, salicylic acid is employed as the at least one anti-acne active in the keratolytic composition.

Optionally, the keratolytic composition may also comprise additional actives acting as boosters for the activity of the above-disclosed anti-acne actives. Suitable additional actives may include: (i) α-hydroxy acids, such as for example citric, lactic, glycolic, malic, tartaric or mandelic acids; (ii) urea; (iii) aminosulfonic compounds, such as for example, N-(2-hydroxyethyl)piperazine-N′-2-ethanesulfonic acid (HEPES); derivatives of 2-oxothiazolidine-4-carboxylic acid, such as for example, procysteine; and their mixtures. Glycolic acid is a preferred additional active for use in the keratolytic composition.

The at least one anti-acne agent may be present in an amount sufficient to stimulate cellular turn-over, and to purify and unclog pores. The anti-acne agent may preferably be present in the keratolytic composition in an amount from at least 0.1 to 20%, preferably from 1 to 15%, and more preferably from 3 to 10% by weight, based on the total weight of the keratolytic composition.

The keratolytic composition may be applied onto the skin in the form of a solution, lotion, gel, fluid or cream.

The Skin Soothing Composition

The skin soothing composition is intended to soothe and protect the skin by forming a barrier that will relieve the skin. The skin soothing composition makes it possible to protect the skin, weakened previously, with respect to further assaults, in particular from the environment.

The skin soothing composition according to the invention will contain at least one skin soothing agent conventionally used to soothe skin. Examples thereof include, but are not limited to, emollients such as silicones, and hydrating agents such as polyols, i.e. glycerin and/or propylene glycol which may be homogenized together in the presence of an emulsifier. It may also comprise additives such as at least one antioxidant for combating free radicals, such as tocopherol, tocopherol acetate, ascorbic acid and arginine pyrrolidonecarboxylate.

The skin soothing agent will typically be present in the composition in an amount of from 1 to 40% by weight, preferably from 3 to 35% by weight, and more preferably from 5 to 25% by weight, based on the weight of the composition. A particularly preferred skin soothing agent is silicone.

The skin soothing composition is generally used in the form of a cream and may be advantageously dispensed from a flexible tube, a pot or any other suitable type of container.

The Maintenance Composition

The maintenance composition contains at least one agent chosen from skin peeling agents and anti-acne agents, and will preferably be milder on the skin than the skin preparatory composition and/or the keratolytic composition.

For some people, the application of the skin preparatory and keratolytic compositions may result in excessive skin irritation. It may then be suggested to skip the application of these compositions every other day and instead apply a maintenance composition. The maintenance composition preferably contains both a skin peeling agent and an anti-acne agent in concentrations such that the maintenance composition, while being effective as an anti-acne composition, will be milder on the skin.

In an embodiment of the invention, the maintenance composition may further contain sunscreens. In a particularly preferred embodiment, the maintenance composition contains 0.3% of capryloyl salicylic acid and 1.5% salicylic acid, all weights based on the weight of the maintenance composition.

The Photoprotective Composition

It is highly recommended that a photoprotective composition, preferably one having an SPF of at least 15, be applied onto treated skin in order to protect it from the harmful effects of the sun.

Suitable organic photoprotective agents may be chosen from anthranilates; cinnamic derivatives; dibenzoylmethane derivatives; salicylic derivatives; camphor derivatives; triazine derivatives, such as those disclosed in patents and patent applications U.S. Pat. No. 4,367,390, EP 863 145, EP 517 104, EP 570 838, EP 796 851, EP 775 698, EP 878 469, EP 933 376, EP 507 691, EP 507 692, EP 790 243 or EP 944 624, the entire contents of which are incorporated herein by reference; benzophenone derivatives; β,β-diphenylacrylate derivatives; benzotriazole derivatives; benzalmalonate derivatives; benzimidazole derivatives; imidazolines; bisbenzoazolyl derivatives, such as disclosed in Patents EP 669 323 and U.S. Pat. No. 2,463,264, the entire contents of which are incorporated herein by reference; p-aminobenzoic acid (PABA) derivatives; methylenebis(hydroxyphenylbenzotriazole) derivatives, such as disclosed in patents and patent applications U.S. Pat. Nos. 5,237,071, 5,166,355, GB 2,303,549, DE 19 726 184 and EP 893 119, the entire contents of which are incorporated herein by reference; benzoxazole derivatives, such as disclosed in Patent Applications EP 832 642, EP 1 027 883, EP 1 300 137 and DE 10 162 844; screening polymers and screening silicones, such as those disclosed in particular in patent application WO 93/04665, the entire contents of which are incorporated herein by reference; dimers derived from α-alkylstyrene, such as those disclosed in patent application DE 19 855 649, the entire contents of which are incorporated herein by reference; 4,4-diarylbutadienes, such as disclosed in Applications EP 967 200, DE 19 746 654, DE 19 755 649, EP 1 008 586, EP 1 133 980 and EP 1 133 981, the entire contents of which are incorporated herein by reference, and their mixtures.

The organic photoprotective agents more particularly preferred may be chosen from the following compounds (CTFA names or chemical names):

Ethylhexyl Salicylate,

Ethylhexyl Methoxycinnamate,

Octocrylene,

Phenylbenzimidazole Sulfonic Acid,

Benzophenone-3,

Benzophenone-4,

Benzophenone-5,

4-Methylbenzylidene Camphor,

Terephthalylidene Dicamphor Sulfonic Acid,

Disodium Phenyl Dibenzimidazole Tetrasulfonate,

2,4,6-Tris(diisobutyl 4′-aminobenzalmalonate)-s-triazine,

Anisotriazine,

Ethylhexyl Triazone,

Diethylhexyl Butamido Triazone,

Methylene Bis-Benzotriazolyl Tetramethyl-butylphenol,

Drometrizole Trisiloxane,

Polysilicone-15,

1,1-Dicarboxy (2,2′-dimethylpropyl)-4,4-diphenylbutadiene,

2,4-Bis[5-1(dimethylpropyl)benzoxazol-2-yl-(4-phenyl)imino]-6-(2-ethylhexyl)imino-1,3,5-triazine, and their mixtures.

Suitable inorganic photoprotective agents may be chosen from pigments or alternatively nanopigments (mean size of the primary particles: generally between 5 nm and 100 nm, preferably between 10 nm and 50 nm) formed of metal oxides which may or may not be coated, such as, for example, nanopigments formed of titanium oxide (amorphous or crystalline in the rutile and/or anatase form), iron oxide, zinc oxide, zirconium oxide or cerium oxide, which are all UV photoprotective agents well known per se. Furthermore, conventional coating agents are alumina and/or aluminum stearate. Such nanopigments formed of metal oxides, which may or may not be coated, are disclosed in particular in Patent Applications EP 518 772 and EP 518 773, the entire contents of which are incorporated herein by reference.

The photoprotective agents can be present in the photoprotective composition in amounts ranging from 2 to 35% by weight, preferably from 5 to 30% by weight, based on the weight of the photoprotective composition. The photoprotective composition preferably has an SPF value of at least 15, such as 20 or 30 or greater.

The compositions contained in the various units of the multi-unit receptacle according to the invention may be provided in all the dosage forms conventionally used for a topical application and in particular in the form of dispersions of the lotion or gel type, of emulsions with a liquid or semi-liquid consistency of the milk type, obtained by dispersion of a fatty phase in an aqueous phase (O/W) or vice versa (W/O), of suspensions or emulsions with a soft, semi-solid or solid consistency of the cream or gel type, of multiple emulsions (W/O/W or O/W/O), of microemulsions, of vesicular dispersions of ionic and/or nonionic type, or of wax/aqueous phase dispersions. These compositions are prepared according to methods known to those of ordinary skill in the art of cosmetics or dermatological formulations.

The oils present in these emulsions can be silicone oils, which may be volatile or nonvolatile, hydrocarbon oils or vegetable oils. These emulsions can additionally comprise non-oily fatty substances, such as shea butter, silicone gums, esters of fatty acids and of fatty alcohols, fatty acids and fatty alcohols.

These compositions can additionally comprise various adjuvants commonly used in the cosmetics field, such as emulsifiers, including glyceryl fatty acid esters, sugar fatty acid esters, sorbitan fatty acid esters, polyethylene glycol fatty acid esters, ethoxylated fatty alcohols and alkylpolyglycosides; fillers, in particular polyacrylamide (Nylon) fibers and/or microbeads, silica, optionally in the form of a colloidal dispersion, and/or organic microspheres which are optionally expanded; preservatives and/or copreservatives, such as caprylyl glycol; sequestering agents, such as EDTA salts; colorants; fragrances; pH adjusters, such as neutralizing agents and/or buffering agents; ethanol; and thickening and gelling agents, in particular acrylamide homo- and co-polymers, acrylic homo- and co-polymers, acrylamidomethylpropanesulphonic acid (AMPS) homo- and co-polymers, and xanthan gum.

Of course, a person skilled in the art will take care to chose this or these possible additional compounds and/or their amounts so that the advantageous properties of the compositions according to the invention are not, or not substantially, detrimentally affected by the envisaged addition.

In order to enhance soothing and/or to lessen irritation, the skin soothing composition and/or the maintenance compositions may be formulated with thermal or mineral waters. By “thermal or mineral water” is meant water having a mineral content of at least 300 mg/l.

In this respect, the term “mineral content” is understood to mean the sum of the concentrations of anions and of cations present in the thermal or mineral water. The fact of using a water with a high mineral content makes it possible to compensate for the irritating effect of the anti-acne and the keratolytic compositions employed previously.

In the present invention, use is made without distinction of a thermal water or of a mineral water. Generally, a mineral water is suitable for consumption, which is not always the case with a thermal water. Each of these waters comprises, inter alia, trace elements and dissolved minerals.

The thermal and/or mineral water used according to the invention can have a mineral content of at least 400 mg/l, in particular of at least 700 mg/l, and more particularly a total concentration of carbonates and of bicarbonates of at least 150 mg/l and more preferably of at least 360 mg/l and in particular of sodium carbonate and bicarbonate of greater than 2 mg/l. The concentration of silicon oxide in the water used in the composition according to the invention can preferably be at least 6 mg/l and more preferably at least 9 mg/l.

The thermal water or the mineral water used according to the invention can be chosen from water from Avène, water from Vittel, waters from the Vichy basin, water from Uriage, water from La Roche Posay, water from La Bourboule, water from Enghien-les-Bains, water from Saint Gervais-les-Bains, water from Néris-les-Bains, water from Allevard-les-Bains, water from Digne, water from Maizières, water from Neyrac-les-Bains, water from Lons-le-Saunier, water from Eaux-Bonnes, water from Rochefort, water from Saint Christau, water from Les Fumades and water from Tercis-les-Bains.

Among these waters, those which exhibit a mineral content of less than 700 mg/l but of greater than 400 mg/l are water from La Roche Posay, water from Eaux-Bonnes or water from Saint Christau.

Among these waters, those which exhibit a total concentration of carbonates or bicarbonates of greater than 360 mg/l are water from Vittel, water from La Bourboule, water from Les Fumades, water from Enghien-les-Bains, water from La Roche Posay, water from the Vichy basin or water from Uriage.

Among these waters, those which exhibit a concentration of carbonates or bicarbonates of between 150 mg/l and 360 mg/l are water from Digne, water from Maizières, water from Rochefort or water from Saint Gervais-les-Bains.

Among these waters, those which comprise at least 2 mg/l of sodium carbonate or bicarbonate are water from La Roche Posay, water from Vittel, waters from the Vichy basin and water from Uriage.

The waters comprising at least 9 mg/l of silicon oxide are water from La Roche Posay, water from Vittel, waters from the Vichy basin or water from Uriage.

Waters from the Vichy basin are preferred for use in the present invention.

The water used in the skin soothing composition and/or the maintenance composition may be water suitable for use in cosmetic or dermatological compositions, which may be exclusively or partially a mineral or thermal water as defined above. The skin soothing composition and/or maintenance composition according to the invention generally may thus comprise no mineral or thermal water, more than 2% by weight, preferably more than 5% by weight, more preferably more than 7% by weight, indeed even make up all the water contained in the skin soothing composition and/or maintenance composition.

The present invention also provides for a method of treating acne on the skin using the above described kit, involving the steps of:

(a) providing a unit containing a skin preparatory composition containing at least one skin peeling agent;

(b) providing a unit containing a keratolytic composition containing at least one anti-acne agent;

(c) providing a unit containing a skin soothing composition containing at least one skin soothing agent;

(d) providing a unit containing a photoprotective composition, followed by, in succession;

(e) contacting the skin with the skin preparatory composition;

(f) applying the keratolytic composition over the skin preparatory composition;

(g) applying a layer of skin soothing composition over the skin preparatory composition and the keratolytic composition;

(h) allowing the skin preparatory composition, keratolytic composition and skin soothing composition to remain on the skin for a predetermined period of time;

(i) removing the skin preparatory composition, keratolytic composition, and skin soothing composition from the skin; and

(j) applying the photoprotective composition onto the skin.

This method is used for an amount of time sufficient for treating the acne-effected area. The precise period of time, i.e. number of days, over which the method will be used will depend upon a number of variables including, but not limited to, the concentration of actives contained in the compositions, as well as the desired effect being sought. However, in all instances, said period of time will be apparent to those skilled in the art.

According to yet another embodiment of the present invention there is provided a modified cosmetic kit containing: (a) a plurality of single-dose units, preferably 11, of 4 ml-each containing a skin preparatory composition having 0.5% capryloyl salicylic acid and 1.5% salicylic acid; (b) a unit containing a keratolytic composition with 3.5% by weight of glycolic acid and 2% by weight of salicylic acid; (c) a dropper for dispensing the keratolytic composition; (d) a unit containing a skin soothing composition; and (e) a unit containing a maintenance composition with 0.3% capryloyl salicylic acid and 1.5% salicylic acid.

The cosmetic kits and methods of the present invention may be used on either a daily basis or on alternate days depending on the concentration of ingredients contained in the kit and/or the desired skin treating application. A particularly preferred period of use is 21 days. Variations thereof, however, will be apparent to those of ordinary skill of the art.

In order to minimize the irritation potential associated with the use of the cosmetic kit of the present invention, it is preferred to use the maintenance composition by itself, on alternate days, in place of the other components of the cosmetic kit. Thus, in use, a person may apply all of the components of a cosmetic kit with the exception of the maintenance composition on days 1, 3, 5, . . . , and use the maintenance composition by itself on days 2, 4, 6, . . . , over a period of 21 days. Daily use of the photoprotective composition is strongly recommended.

The present invention will be better understood from the examples which follow, all of which are intended for illustrative purposes only and are not meant to unduly limit the scope of the invention in any way.

The Skin Preparatory Composition

A skin preparatory composition in accordance with the present invention was prepared having the following formulation:

| Ingredient (CTFA Designation) | % w/w |
|---|---|
| PEG-6 | 3.00 |
| Glycerin | 4.00 |
| Salicylic Acid | 1.50 |
| Water | 5.00 |
| Capryloyl Salicylic Acid | 0.50 |
| Alcohol Denat. | q.s 100 |

The skin preparatory composition was applied in the evening onto a face having acne thereon, avoiding the outline of the eyes and lips, by massaging the face with a cotton pad moistened with said solution for about 1 minute to 2 minutes. The skin preparatory composition made it possible to exfoliate the top layer of skin, correct surface imperfections, and increase epidermal permeability. It prepared the skin for the application of the keratolytic composition.

The Keratolytic Composition

A keratolytic composition in accordance with the present invention was prepared having the following formulation:

| Ingredient (CTFA Designation) | % w/w |
|---|---|
| Salicylic Acid | 2.00 |
| Water | 11.50 |
| Glycolic Acid | 3.50 |
| Acrylates/Octylacrylamide Copolymer* | 0.50 |
| Hydroxypropylcellulose | 0.85 |
| PPG-12/SMDI Copolymer** | 1.00 |
| Alcohol Denat. | q.s 100 |

*Available from National Starch as DERMACRYL LT
**Available from Bertek Pharmaceuticals as POLYOLPREPOLYMER-2

The pH of this solution was adjusted to 3.5 to 4.0 by using a 10% w/w solution of sodium hydroxide.

The keratolytic solution was applied onto the face, over the skin preparatory composition using a dropper of a capacity of 0.5 ml. Three droppers full were sufficient for application to the entire face, while avoiding the outline of the eyes and lips. The keratolytic solution improved cell renewal and treated the acne condition.

The Skin Soothing Composition

A skin soothing composition in accordance with the present invention was prepared having the following formulation:

| Ingredients (CTFA Designation) | % w/w |
|---|---|
| PEG/PPG-18/18 Dimethicone | 1.00 |
| C30-45 Alkyl Dimethicone | 3.00 |
| Preservatives | 1.00 |
| Cyclopentasiloxane | 24.50 |
| Glycerin | 7.00 |
| Propylene Glycol | 3.00 |
| Dimethicone | 1.50 |
| Cetyl Dimethicone | 3.00 |
| Magnesium Sulfate | 1.00 |
| Polysorbate 20 | 0.50 |
| Water | q.s 100 |

The pH of the internal aqueous phase was 5.50.

The skin soothing composition was applied over the skin preparatory composition and the keratolytic composition and left on the face overnight.

The Maintenance Composition

In order to minimize the irritation potential of the above-described compositions, a maintenance composition was used, on alternate days, in place of the above-described compositions.

A maintenance composition in accordance with the present invention was prepared having the following formulation:

| Ingredients (CTFA Designation) | % w/w |
|---|---|
| Fragrance | 0.30 |
| PEG-4 Laurate | 0.08 |
| Xanthan Gum | 0.20 |
| PEG-4 | 0.02 |
| Glyceryl Stearate | 2.50 |
| Sodium Hydroxide | 0.72 |
| Di-C12-13 Alkyl Malate | 10.00 |
| Zinc PCA | 0.10 |
| Cetyl Alcohol | 1.00 |
| Cyclohexasiloxane | 5.00 |
| Iodopropynyl Butylcarbamate | 0.02 |
| Propylene Glycol | 5.00 |
| Salicylic Acid | 1.50 |
| Capryloyl Salicylic Acid | 0.30 |
| PEG-100 Stearate | 2.50 |
| PEG-4 Dilaurate | 0.08 |
| Aluminum Starch Octenylsuccinate | 3.00 |
| Acrylates/C10-30 Alkyl Acrylate Crosspolymer | 0.70 |
| Water | q.s 100 |

The pH was adjusted to a value of from about 5.5 to about 6.1.

After a period of use spanning 21 days, a noticeable improvement in the acne condition was observed.

What is claimed is:

1. A cosmetic kit for treating human skin comprising a multi-unit receptacle containing:
(a) at least one unit containing a skin preparatory composition having at least one skin peeling agent;
(b) at least one unit containing a keratolytic composition having at least one anti-acne agent;
(c) at least one unit containing a skin soothing composition having at least one skin soothing ingredient;
(d) optionally, at least one unit containing a maintenance composition having at least one agent chosen from a skin peeling agent and an anti-acne agent;
(e) optionally, at least one unit containing a photoprotective composition.

2. The kit of claim 1 wherein the at least one skin peeling agent is a hydroxy acid.

3. The kit of claim 1 wherein the at least one skin peeling agent is capryloyl salicylic acid.

4. The kit of claim 1 wherein the at least one skin peeling agent is present in an amount of from about 3 to about 20% by weight, based on the weight of the skin preparatory composition.

5. The kit of claim 1 wherein the skin preparatory composition further includes at least one anti-acne agent.

6. The kit of claim 1 wherein the at least one anti-acne agent present in the keratolytic composition is salicylic acid.

7. The kit of claim 1 wherein the keratolytic composition contains an anti-acne agent in an amount of from about 0.1 to about 20% by weight, based on the weight of the keratolytic composition.

8. The kit of claim 1 wherein the keratolytic composition further contains at least one skin peeling agent.

9. The kit of claim 1 wherein the at least one agent of the maintenance composition is present in an amount less than that of (a) and/or (b).

10. The kit of claim 1 wherein the at least one agent of the maintenance composition is a skin peeling agent.

11. The kit of claim 1 wherein the at least one agent of the maintenance composition is an anti-acne agent.

12. The kit of claim 1 wherein the at least one agent of the maintenance composition is a skin peeling agent and an anti-acne agent.

13. The kit of claim 1 wherein the photoprotective composition has an SPF value of at least about 15.

14. The kit of claim 1 further comprising at least one water chosen from thermal water and mineral water, and wherein the at least one water is present in the skin soothing composition and/or the maintenance composition.

15. A method of treating acne, comprising:
(a) providing a unit containing a skin preparatory composition having at least one skin peeling agent;
(b) providing a unit containing a keratolytic composition having at least one anti-acne agent;
(c) providing a unit containing a skin soothing composition having at least one skin soothing agent;
(d) optionally, providing a unit containing a maintenance composition having at least one agent chosen from skin peeling agents and anti-acne agents;
(e) optionally, providing a unit containing a photoprotective composition;
(f) contacting the skin with the skin preparatory composition;
(g) applying the keratolytic composition over the skin preparatory composition;
(h) applying the skin soothing composition over the skin preparatory composition and the keratolytic composition;
(i) allowing the skin preparatory composition, keratolytic composition and skin soothing composition to remain on the skin for a predetermined period of time;
(j) removing the skin preparatory composition, keratolytic composition, and skin soothing composition from the skin; and
(k) applying the photoprotective composition onto the skin.

16. The method of claim 15 wherein the method is performed daily.

17. The method of claim 15 wherein the method is performed on alternate days.

18. The method of claim 15 wherein the skin preparatory composition contains at least one type of skin peeling agent chosen from hydroxy acids.

19. The method of claim 15 wherein the at least one skin peeling agent is capryloyl salicylic acid.

20. The method of claim 15 wherein the at least one skin peeling agent is present in an amount of from about 3 to about 20% by weight, based on the weight of the skin preparatory composition.

21. The method of claim 15 wherein the skin preparatory composition further includes at least one anti-acne agent.

22. The method of claim 15 wherein the at least one anti-acne agent present in the keratolytic composition is salicylic acid.

23. The method of claim 15 wherein the keratolytic composition contains an anti-acne agent in an amount of from about 0.1 to about 20% by weight, based on the weight of the keratolytic composition.

24. The method of claim 15 wherein the keratolytic composition further contains at least one skin peeling agent.

25. The method of claim 15 wherein the at least one agent of the maintenance composition is present in an amount less than that of (a) and/or (b).

26. The method of claim 15 wherein the at least one agent of the maintenance composition is a skin peeling agent.

27. The method of claim 15 wherein the at least one agent of the maintenance composition is an anti-acne agent.

28. The method of claim 15 wherein the at least one agent of the maintenance composition is a skin peeling agent and an anti-acne agent.

29. The method of claim 15 wherein the photoprotective composition has an SPF of at least about 15.

30. The method of claim 15 further comprising providing at least one water chosen from thermal water and mineral water, and wherein the at least one water is present in the skin soothing composition and/or the maintenance composition.

31. The method of claim 15 wherein only the maintenance composition is applied onto the acne on the alternate days.

32. A cosmetic kit for treating human skin comprising a multi-unit receptacle containing:
(a) a plurality of 4 ml single-dose units containing a skin preparatory composition having about 0.5% by weight of capryloyl salicylic acid and about 1.5% by weight of salicylic, all weights based on the weight of the skin preparatory composition;
(b) a unit containing a keratolytic composition having about 3.5% by weight glycolic acid and about 2% salicylic acid by weight, all weights based on the weight of the keratolytic composition;
(c) a dropper for dispensing the keratolytic composition;
(d) a unit containing a skin soothing composition having at least one skin soothing agent; and
(e) a unit containing a maintenance composition containing about 0.3% by weight of capryloyl salicylic acid and about 1.5% by weight of salicylic acid, all weights based on the weight of the maintenance composition.

33. The kit of claim 32 further comprising at least one water chosen from thermal water and mineral water, and wherein the at least one water is present in the skin soothing composition and/or the maintenance composition.
